(19) United States
(12) Patent Application Publication
(10) Pub. No.: US 2007/0243200 A1
(43) Pub. Date: Oct. 18, 2007

(54) Trehalose producing cells as vaccines

(75) Inventor: Camilo Anthony Leo Selwyn Colaco, Cambridge (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: ImmunoBiology Limited, Babraham (GB)

(21) Appl. No.: 11/807,855

(22) Filed: May 29, 2007

Related U.S. Application Data
(62) Division of application No. 10/049,706, filed on Apr. 16, 2002, now abandoned.

(30) Foreign Application Priority Data
Aug. 19, 1999 (GB) 9919732.9

(51) Int. Cl. A61K 39/395; C12N 1/21; C12P 1/04

(52) U.S. Cl. 424/184.1; 435/243; 435/71.1

(57) ABSTRACT

The present invention relates to methods for using prokaryotic cells which have been modified or induced to synthesize trehalose as vaccines and to vaccine compositions obtained thereby.

### CROSS-REFERENCE TO RELATED APPLICATION

This is a divisional of co-pending application Ser. No. 10/049,706, filed Apr. 16, 2002, which is a 371 of PCT/GB00/03223, filed Aug. 18, 2000.

### FIELD OF THE INVENTION

This invention relates to the field of vaccines. More specifically, it relates to methods of producing vaccines of trehalose containing procaryotic cells and the compositions obtained thereby.

### BACKGROUND TO THE INVENTION

Procaryotic cells, particularly bacteria, are widely and increasingly used in medical, agricultural and industrial applications. Agricultural, or environmental, applications include biopesticides and bioremediation.

Medical applications include use of bacteria in vaccines as well as for production of pharmaceutical products for other treatments.

For the procaryotic cells to be used effectively, both in terms of desired results and cost, the cells must be able to be stored for significant periods of time whilst preserving their viability. The term viability is used herein to denote that the cells manifest the features of a functioning living organism, such as metabolism and cell division.

Methods for preserving live procaryotic cells suffer from several serious drawbacks, such as being energy-intensive and requiring cold storage. Thus, freeze-drying is often used for preservation and storage of procaryotic cells.

However, it has the undesirable characteristic of significantly reducing viability of the cells, as well as being time-and energy-intensive and thus expensive.

PCT GB97/03375 describes a process of stabilising procaryotic cells by the induction of trehalose synthesis and the drying of the resulting cells in a glassy carbohydrate matrix. This process gives stabilised cells that can be stored at ambient temperatures without loss of viability. Trehalose,(α-D-glucopyranosyl-α-D-glucopyranoside), is a naturally occurring, non-reducing disaccharide which was initially found to be associated with the prevention of desiccation damage in certain plants and animals which can dry out without damage and can revive when re-hydrated. Trehalose has been shown to be useful in preventing denaturation of proteins, viruses and foodstuffs during desiccation, see U.S. Pat. Nos. 4,891,319; 5,149,653; 5,026,566; Colaco et al. (1992) Bio/Tech. 10: 1007-1011.

Trehalose synthesis in procaryotic cells is induced by a number of methods including osmotic shock which induces the endogenous production of trehalose, Welsh et al. (1991) J. Gen. Microbiol. 137: 745-750.

PCT application No. GB94/01556 describes a process of improving the viability of bacterial dried cells by the induction of trehalose synthesis by nutrient limitation, heat shock or osmoadaptation. PCT application No. GB97/03375 describes a method for the preservation of procaryotic cells by the drying of cells in a carbohydrate matrix after the induction of trehalose synthesis. The latter invention provides compositions of dried cells that can be stored at ambient temperatures and thus enable a number of industrial applications.

Surprisingly, we have now found that the dried, stabilised procaryotic cells produced by the above methods, are more immunogenic than fresh live cells and hence have particular value as the immunogenic determinant active component in vaccine compositions. Furthermore, we have also found that this increased immunogenicity of the stabilised procaryotic cells is not dependent on the drying process considered essential in the above stabilisation processes, but results from the induction of trehalose synthesis. Although more pronounced with dried cells, this increased immunogenicity is also seen in cells induced to produce trehalose but which have not been subjected to a drying process.

### SUMMARY OF THE INVENTION

The present invention thus provides a method for producing a vaccine composition, which comprises the steps of:

a. Treating procaryotic cells in vitro under conditions such that an increase of the concentration of trehalose within procaryotic cells is induced, preferably by the synthesis of trehalose within the cell;

b. using the induced cells containing trehalose as the immunogenic determinant in the production of a vaccine composition.

Preferably, the treatment of the procaryotic cells is carried out to achieve a concentration of trehalose within the cells of at least 10 mM.

Preferably, the induced cells containing the trehalose are dried prior to their use in the production of the vaccine composition, notably in the presence of a non-reducing carbohydrate such as trehalose to provide a storage stable but viable immunogenic determinant for storage prior to use in a vaccine composition.

The invention also provides a vaccine composition containing an immunogenic determinant, characterised in that the immunogenic determinant has been made by the method of the invention.

The invention also provides a method for immunizing an animal which comprises administering a pharmaceutically effective amount of a vaccine composition of the invention to an animal sufficient to elicit an immune response in the animal.

Preferably, the vaccine composition contains an adjuvant for the immunogenic determinant, is put up in an aqueous carrier medium and is administered by injection.

The procaryotic cells for use in the present invention are ones which are capable of synthesising trehalose. This ability can be native or can be conferred by recombinant techniques. The ability of a procaryotic cell to synthesise trehalose can be determined by measuring trehalose concentration as described below.

The term procaryotic is used herein to denote cells that exhibit characteristics of procaryotes, which are typically unicellular organisms, lack organelles (such as mitochondria, chloroplasts, and Golgi apparatus), lack a cytoskeleton and lack a discrete nucleus. Examples of procaryotic cells for present use include bacteria, such as eubacteria, cyanobacteria and prochlorophytes; archaebacteria; and other microorganisms such as rickettsias, mycoplasmas, spiroplasmas, and chlamydiae.

Preferred procaryotic cells for present use are bacteria.

In general, any procaryotic cell or mixture of cells, particularly bacteria, containing trehalose synthase genes should be capable of synthesising trehalose. Bacteria have two genes involved in trehalose synthesis (i.e. T Phosphate synthase and T-P phosphatase), whereas yeasts have at least three genes and combinations of these genes may be used to enable trehalose synthesis. Examples of bacteria that contain the trehalose synthase gene include, but are not limited to, Enterobacteriaceae, such as Salmonella and Escherichia (e.g., S. typhimurium and E.coli); halophilic and halotolerant bacteria, such as Ectothriorhodospira (e.g., E. halochloris) ; micrococco-caceae, such as Micrococcus (e.g., M. luteus); Rhizobium species such as R. japonicum and R. leguminosarum bv phaseoli; Cyanobacteria; Mycobacteria species such as M. tuberculosis, M.bovis, and M. smegmatis.

Procaryotic cells can be induced to synthesise trehalose by culturing the cells in vitro under stressful conditions, e.g., osmotic shock, heat or oxygen limitation (shock), carbon/nitrogen starvation, or any combination of the above. Suitable conditions include those heat shock and other conditions described, for example, in PCT applications Nos. GB94/01556 and GB97/03375.

Alternatively, use of inhibitors, such as validomycin, of enzyme (s) such as trahalase involved in trehalose degradation may also result in an increase of trehalose concentration within the cells. Alternatively, the genetic structure of the procaryotic organism may be modified to remove or inhibit that portion of the genetic structure which inhibits or restricts the synthesis of trehalose within the cell so that the cells constitutively synthesise trehalose as they are cultured without the need to apply external stimuli. Such genetic modification can be achieved using any suitable technique. For convenience, the invention will be described hereinafter in terms of the use of external stimuli to induce the production of trehalose within the cell, rather than the use of a procaryotic cell which has had its genetic structure modified.

The term osmotic shock is used herein to denote that the solute concentration in the growth medium within which the cells are cultivated is above the level at which a cell exists and/or grows in its native environment. The solute may be a mixture of salts and the concentration is typically from 0.2 to 0.5 Mols above the level at which the cell is normally cultivated.

We believe that induction of trehalose synthesis under stressful conditions may also induce synthesis or accumulation of other molecules that may be beneficial for preservation, such as betaine and chaperonins or which enhance the vaccine action of the induced cells.

For bacteria, particularly Escherichia, trehalose synthesis is preferably induced by growing the cell (s) in conditions of high osmolarity, i.e., salt concentrations sufficient to stimulate trehalose production. To induce trehalose synthesis by osmotic shock, the total concentration of salt (s) in the medium should be at least about 0.2M, preferably at least about 0.4M, more preferably at least about 0.5M. The total concentration of salt (s) should not exceed 0.6M, since above this level trehalose synthesis declines in E. coli. The salt concentrations correspond to osmolarities of at least about 350 mosmoles to about 1.5 Osmoles, preferably at least about 400 mOsmoles to 1 Osmole, most preferably 250 mOsmoles to 500 mOsmoles. Generally, a minimum osmolarity of about 200 mOsmoles is required as this will usually provide a higher concentration of solute than that under which the cells are usually cultivated.

The necessary solute can be provided by the use of a single salt, for example, 200 mM NaCl KCl and/or CaCl₂. (NH₄)₂SO₄ may also be used, however only about one half of the amount of trehalose is produced compared to that produced in the presence of KCl, NaCl and/or CaCl₂. A mixture of salts can also be used. In addition, when used to increase the osmolarity of the medium, a non-penetrant solute such as sorbitol and/or glucose can contribute to the stimulation of trehalose synthesis.

The salt concentration (i.e., osmolarity) required to stimulate and/or induce trehalose synthesis will depend upon the genus, species, and/or strain of the procaryotic cell used. Preferably, cell(s) are grown in a minimal medium containing solutes and commercially available minimal media can be supplemented with desired salts and/or other solutes. The use of a minimal medium is not essential and defined media can also be used. The time required to initiate and achieve the desired level of trehalose concentration within the cells will vary depending on the level of osmolarity as well as the genus, species and/or strain of procaryotic cell used. Trehalose synthesis will generally begin within an hour of placing cells in conditions designed to stimulate trehalose production. Generally, in E. coli the synthesis of trehalose reaches a maximum at about 15-20 hours.

Synthesis of trehalose may also be stimulated using recombinant methods which are well known in the art. For instance, procaryotic cells can be transfected with a DNA plasmid comprising a DNA sequence encoding the appropriate trehalose synthase gene. The gene in turn is operatively linked to a suitable promoter, which can be constitutive or inducible. Suitable recombinant techniques are described in, for example, Molecular Cloning: A Laboratory Manual, second edition (Sambrook et al., 1989).

The concentration of trehalose synthesised within the procaryotic cells can be measured using any suitable assay technique, for example by high pressure liquid chromatography (HPLC), coupled with electrochemical detection and glucose assay (Trinder assay using trehalase) for quantitative enzymatic determination of trehalose.

Thin layer chromatography can be used as a qualitative method for the separation of different carbohydrates.

Refractive index detection provides another means of detecting sugars quantitatively.

In measuring trehalose by HPLC, cells are disrupted and trehalose preferentially solubilized in 70% ethanol, followed by removing triglycerides by chloroform extraction. Trehalose concentration is determined by multiplying trehalose concentration (as determined by a standard curve) by the fraction of final volume of supernatant divided by pellet volume. A more detailed description of this assay is provided in Example 1.

Preferably, the synthesis is carried out to provide a concentration of trehalose within the cells of at least about 10 mM, for example at least about 30 mM, preferably at least about 50 mM, notably at least about 100 mM.

Thus, in a preferred aspect the invention includes culturing the procaryotic cells under conditions that stimulate intracellular production of trehalose, wherein intracellular concentration of trehalose reaches at least about 10 mM, preferably at least about 30 mM, more preferably at least about 50 mM, notably at least about 100 mM. It is particularly preferred that the concentration be at least about 150 mM.

The time required for stimulating trehalose synthesis depends, inter alia, on the nature of the procaryotic cells (including genus, species, and/or strain) and the conditions under which trehalose induction occurs (i.e., whether by osmotic shock, oxygen deprivation, etc.). For trehalose induction by osmotic shock, the time required for maximum concentration of trehalose in turn depends on the degree of osmolarity as well as the particular salts used. The optimum conditions for trehalose synthesis can readily be determined by simple trial and errors tests.

The cultivated procaryotic cells containing the intracellular trehalose may then be frozen for storage before use as a vaccine. Alternatively storage of the vaccine can be effected by culturing the procaryotic cells under conditions that increase trehalose concentration to a level effective to increase storage stability, mixing the cells with a drying solution which contains a stabilising agent, and drying the cells under conditions such that a glass is produced having less than about 5% residual moisture. If a killed vaccine rather than a live vaccine is required, the cells may be killed by any suitable method, for example chemical fixation and radiation prior to processing for storage. Though the procaryotic cells may be used as the sole immunogenic determinant active ingredient in the vaccine, an adjuvant may be added in an amount sufficient to enhance the immune response to the procaryotic vaccine. The adjuvant can be added to the procaryotic cells before drying, for example, cholera B toxin sub-unit can be dried simultaneously with V. cholera. Alternatively the adjuvant may be obtained and dried separately, and reconstituted along with the procaryotic cells.

Suitable adjuvants include, but are not limited to, aluminium hydroxide, alum, QS-21 (U.S. Pat. No. 5,057,540), DHEA (U.S. Pat. Nos. 5,407,684 and 5,077,284) and its derivatives (including salts) and precursors (e.g., DHEA S), beta-2 microglobulin (WO 91/16924), muramyl dipeptides, muramyl tripeptides (U.S. Pat. No. 5,171,568), monophosphoryl lipid A (U.S. Pat. No. 4,436,728; WO 92/16231) and its derivatives (e.g., DETOX ), and BCG (U.S. Pat. No. 4,726,947). Other suitable adjuvants include aluminium salts, squalene mixtures (SAF-1), muramyl peptide, saponin derivatives, mycobacterium wall preparations, mycolic acid derivatives, non-ionic block copolymer surfactants, QUIL A™ (Quil A Saponin), cholera toxin B sub-unit, polyphosphazene and derivatives, and immunostimulating complexes (ISCOMs) such as those described by Takahashi et al. (1990) Nature 344: 873-875. The choice of an adjuvant will depend in part on the stability of the vaccine in the presence of the adjuvant, the route of administration, and the regulatory acceptability of the adjuvant, particularly when intended for human use. For instance, alum is approved by the United States Food and Drug Administration (FDA) for use as an adjuvant in humans.

The invention also provides a method for treating an animal with a vaccine of the invention by administering a pharmaceutically acceptable quantity of the vaccine of the invention, optionally in combination with an adjuvant, sufficient to elicit an immune response in the animal.

The animal is typically a human. However, the invention can also be applied to the treatment of other mammals such as horses, cattle, goats, sheep or swine, and to the treatment of birds, notably poultry such as chicken or turkeys.

The vaccine compositions of the present invention may be administered by any suitable means, such as orally, by inhalation, transdermally or by injection and in any suitable carrier medium. However, it is preferred to administer the vaccine as an aqueous composition by injection using any suitable needle or needle-less technique.

The vaccines of the invention may contain any suitable concentration of the induced procaryotic cells. We prefer that the cells are administered at doses in the range of 10-600 μg, preferably 10-100 μg, most preferably 25 μg, per Kg of body weight of the animal being treated. It will be appreciated that the vaccine of the invention may be applied as an initial treatment followed by one or more subsequent treatments at the same or a different dosage rate at an interval of from 1 to 26 weeks between each treatment to provide prolonged immunization against the pathogen.

The following examples are provided to illustrate but not limit the invention.

### EXAMPLE 1

### Induction of Trehalose in E. coli by Osmotic Shock:

E. coli (NCIMB strain 9484) was cultured in Evans medium (pH 7.0) containing 5 mM ammonium chloride. After overnight incubation at 37° C. in the initial Evans medium, a 4 ml culture of E. coli grown in Evans medium under nitrogen limitation was used to inoculate a 200 ml culture of Evans medium modified to induce osmotic shock by increasing the salt concentration (KCl) to 0.5M.

Trehalose concentration was measured by high pressure liquid chromatography (HPLC) analysis and significant increases in trehalose concentrations were observed at 15-17 hours after initiation of osmotic shock, with values peaking at less than 20 hours.

### EXAMPLE 2

### Induction of Trehalose Synthesis in Salmonella:

Salmonella typhimurium (1344) was grown overnight at 37° C. in M9 (minimal) medium with and without 0.5M NaCl. Cells were harvested by centrifugation and analysed for trehalose concentration by HPLC analysis as described in Example 1. Growth in high salt medium showed at 4 to 5 fold induction of trehalose synthesis as compared to the low salt medium.

### EXAMPLE 3

### Drying of Procaryotic Cells after Induction of Trehalose Synthesis:

E. coli and Salmonella typhimurium were grown overnight at 37° C. in M9 (minimal) medium with and without 0.5M NaCl and trehalose synthesis induced as described in examples 1 and 2. The induced bacteria were harvested by centrifugation at 10,000 rpm for 10 minutes and the cell pellets resuspended in drying solution containing 45% trehalose, 0.1% cmc (sodium carboxymethyl cellulose, Blanose 7HF, Aqualon) to a typical cell density of 0.5-1.2×10⁹ CFU/ml. 300 μl and 500 μl aliquots were dispensed into 3 ml pharmaceutical vials and dried under vacuum without freezing, overnight at ambient temperature and a vacuum pressure of 30 mTorr. Alternatively, the aliquots can be freeze-dried using the following protocol: ramp at 2.5° C./min to an initial shelf temperature of −40° C.; primary drying was performed at a vacuum pressure of 30 mT at −40° C. and held for 40 hours; for secondary drying ramp at 0.05° C./min from −40 to 30° C. and hold for 12 hours.

### EXAMPLE 4

### Use of Induced Procaryotic Cells as Vaccines:

E. coli and Salmonella typhimurium cells were induced to synthesise trehalose as in Examples 1 and 2 and were used to immunize mice and rabbits. Titration of the bacteria showed that a 100 to 1000 fold lower titre of bacteria induced for trehalose synthesis was required to produce an equivalent antibody response in the animals compared to the use of non-induced bacteria. Dried preparations were generally 2-50 fold more effective on a cell number basis at eliciting protective immunity in the immunized animals than non-dried preparations.

### EXAMPLE 5

### Use of Induced Procaryotic Cells as Vaccines; Heat-induced Trehalose Synthesis:

E. coli and Salmonella typhimurium (strains as in examples 1 and 2) were grown overnight at 37° C. in LB medium. 4 ml aliquots of the stationary cultures were used to inoculate 200 ml of LB medium in a 2 litre conical flask and the cultures grown for 3 hrs at 30° C. The log phase cultures were then raised to 40° C. and grown for a further 3 hrs before the bacteria were harvested by centrifugation at 10,000 rpm for 10 minutes. A similar protocol was used for the growth and induction of Mycobacterium Bovis and Vaccae (NCTC 11659) which were grown for 2 days in Sauton's medium before dilution to obtain log phase cultures for heat-induction. Cell pellets were resuspended in lysis solution containing 0.5% Tween and the trehalose concentration was measured by high pressure liquid chromatography (HPLC) analysis. Typically 3-5 fold increases in trehalose concentrations were observed as compared to cells grown at 30° C. alone.

Bacterial cells induced to synthesise trehalose as described above were killed by repeated freeze-thaw cycles and used to immunize rabbits. Antibody titres in the immunized animals were assayed by 10-fold serial dilutions using a dot-blot assay on total cell lysates prepared as described for trehalose analysis above. Animals vaccinated with induced bacteria showed a 10 to 100 fold higher antibody titre than those immunized with non-induced bacteria.

1. A method for producing a vaccine composition containing an immunogenic determinant as the active ingredient, the method comprising the steps of:
a. treating prokaryotic cells under conditions such that an increase of the concentration of trehalose within the prokaryotic cells is induced;
b. using the induced cells containing trehalose as the immunogenic determinant in the production of a vaccine composition.

2. The method as claimed in claim 1, wherein the treatment of the prokaryotic cells is carried out to achieve a concentration of trehalose within the cells of at least 10 mM.

3. The method as claimed in claim 1, wherein the increase in concentration of trehalose is achieved by synthesis of trehalose within the cell.

4. The method as claimed in claim 1, wherein the condition causing the increase of trehalose concentration within the cells is heat, osmotic shock, suppression of degradation of trehalose, or genetically engineered constitutive synthesis of trehalose within the cells.

5. The method as claimed in claim 1, wherein the induced cells containing the trehalose are dried prior to their use in the production of the vaccine composition.

6. The method as claimed in claim 5, wherein the cells are dried in the absence of added extra-cellular carbohydrate glassy stabilising matrix.

7. The method as claimed in claim 1, wherein the procaryotic cells are bacteria, protozoa or fungi.

8. The method as claimed in claim 1, wherein the prokaryotic cells are treated by cultivating them in a medium containing one or more solutes and having an osmolarity of at least 350 mOsmoles.

9. The method as claimed in claim 8, wherein the solute is selected from the group consisting of sodium, potassium, calcium and ammonium salts, and combinations thereof.

10. The method as claimed in claim 1, wherein the prokaryotic cell has been modified so as to synthesise trehalose.

11. The method as claimed in claim 1, wherein the treatment of the cells is carried out to achieve a concentration of trehalose within the cells of at least 100 mM.

12. The method as claimed in claim 1, wherein the prokaryotic cells containing the induced trehalose are killed prior to use in the vaccine composition.

13. The method as claimed in claim 1, wherein the treatment of the prokaryotic cells is carried out in vitro.

14. A method for treating an animal with a vaccine, comprising administering to said animal a pharmaceutical effective amount of a vaccine composition as claimed in claim 1 to elicit an immune response in the animal.

15. The method as claimed in claim 14, wherein the vaccine composition is administered by injection.

16. A prokaryotic cell which has had its genetic structure modified so as to remove or inhibit that portion of the genetic structure which inhibits or restricts the synthesis of trehalose by the cell, whereby the cell constitutively synthesises trehalose within the cell as it grows.
